(19) United States
(12) Patent Application Publication
(10) Pub. No.: US 2019/0151290 A1
(43) Pub. Date: May 23, 2019

(54) ALEGLITAZAR FOR THE TREATMENT OF DIABETIC KIDNEY DISEASE

(71) Applicant: Hoffmann-La Roche Inc., Little Falls, NJ (US)

(72) Inventors: Regina DUTTLINGER MADDUX, Basel (CH); Carl Anders SVENSSON, Basel (CH); Jin TIAN, Shanghai (CN); Harold V. BARRON, South San Francisco, CA (US)

(73) Assignee: Hoffmann-La Roche Inc., Little Falls, NJ (US)

(21) Appl. No.: 16/128,892

(22) Filed: Sep. 12, 2018

Related U.S. Application Data
(63) Continuation of application No. PCT/EP2016/077521, filed on Nov. 14, 2016.

(30) Foreign Application Priority Data
Nov. 18, 2015 (CN) PCT/CN2015/094942

(51) Int. Cl. A61K 31/422

(52) U.S. Cl. CPC A61K 31/422

(57) ABSTRACT

The present invention relates to the use of aleglitazar in the treatment or prevention of diabetic kidney disease in a patient having a linear decline in GFR.

### CROSS REFERENCE TO RELATED APPLICATIONS

This application is a Continuation of International Patent Application PCT/EP/2016/077521 filed on Nov. 14, 2016 which is entitled to the benefit of PCT/CN2015/094942 filed on Nov. 18, 2015, the disclosures of which are incorporated herein by reference.

### FIELD OF THE INVENTION

The present invention relates to aleglitazar for use in the treatment or prevention of diabetic kidney disease in a patient having a linear decline in GFR.

### BACKGROUND OF THE INVENTION

Aleglitazar is (S)-2-methoxy-3-{4-[2-(5-methyl-2-phenyl-oxazol-4-yl)-ethoxy]-benzo[b]thiophen-7-yl}-propionic acid. It belongs to the class of Peroxisome Proliferator Activated Receptors (PPAR) agonists. Aleglitazar is described in WO 02/092084.

### SUMMARY OF THE INVENTION

The invention further relates to Aleglitazar for use in the stabilization of kidney function as described herein.

### BRIEF DESCRIPTION OF THE DRAWINGS

FIG. 1 represents the eGFR slope analysis for patients with baseline eGFR inferior to 80 mL/min/1.73 m² and UACR superior to 300 mg/mg.

### DETAILED DESCRIPTION OF THE INVENTION

Peroxisome Proliferator Activated Receptors (PPAR) are members of the nuclear hormone receptor super family, which are ligand-activated transcription factors regulating gene expression. Various subtypes thereof have been identified and cloned. These include PPAR alpha, PPAR beta (also known as PPAR delta) and PPAR gamma. There exist at least two major isoforms of PPAR gamma. While PPAR gamma1 is ubiquitously expressed in most tissues, the longer isoform PPAR gamma2 is almost exclusively found in adipocytes. In contrast, PPAR alpha is predominantly expressed in the liver, kidney and heart. PPARs modulate a variety of body responses including glucose- and lipid-homeostasis, cell differentiation, inflammatory responses and cardiovascular events.

Aleglitazar is a potent, balanced dual PPAR alpha/gamma agonist. It combines the PPAR alpha with the PPAR gamma agonist effects.

In the randomized clinical trial AleCardio (Effect of Aleglitazar on Cardiovascular Outcomes After Acute Coronary Syndrome in Patients With Type 2 Diabetes Mellitus), although aleglitazar reduced hemoglobin A1C and improved serum HDL-C and triglyceride levels, consistently with other previous PPAR studies, it did not significantly decrease the incidence of cardiovascular death, myocardial infarction or stroke in the overall population. Additionally, aleglitazar treatment showed increased risks of heart failure, renal dysfunction, bone fracture, GI bleeding and hypoglycemia.

However, aleglitazar surprisingly demonstrated in this study a positive effect on the stabilization of renal function in patients who had a linear decrease in GFR (glomular filtration rate).

AleCardio was a randomized, double-blind, placebo controlled, multicenter study to evaluate the effect of aleglitazar on cardiovascular outcomes after acute coronary syndrome (ACS) in patients with type 2 diabetes mellitus. The study design has previously been published in Lincoff et al., JAMA 2014 and Lincoff et al., Am Heart J. 2013; 166(3):429-434. The study protocol was approved by the institutional review board of each center and all patients gave written informed consent. The study was overseen by steering and safety committees. The steering committee oversaw the study design, the study conduction and the study data analysis. A Data and Safety Monitoring Board (DSMB), consisting of independent physicians and statisticians with access to unblinded data, monitored the safety of the study. 7,226 patients who were hospitalized for ACS with either established or newly diagnosed type 2 diabetes were recruited between February 2010 and May 2012 from 722 centers in 26 countries. The study was planned to continue until patients were followed-up for at least 2.5 years and 950 primary end point events were positively adjudicated. However, the trial was terminated in July 2013 following the DSMB's recommendation and 704 primary end points events (74% of those projected) had been positively adjudicated by Dec. 17, 2013.

The study enrolled patients with type 2 diabetes mellitus and acute coronary syndrome (ACS) requiring hospitalization. Acute coronary syndrome included myocardial infarction, with or without ST segment elevation on the electrocardiogram or biomarker-negative unstable angina. Exclusion criteria included symptomatic heart failure, hospitalization with heart failure within the previous 12 months, severe peripheral edema, estimated glomerular filtration rate of <45 mL/min/1.73 m² or fasting triglyceride level greater than 400 mg/dL. Patients could be randomized at hospital discharge following the qualifying ACS event or after a screening period of no longer than 12 weeks to allow stabilization of their clinical condition, completion of planned revascularization procedures and achievement of steady-state renal function.

Patients were assigned in a double-blind fashion under a 1:1 ratio using a permuted block randomization without stratification through an interactive telephone and web system to receive aleglitazar 150 μg daily or matching placebo, in addition to contemporary and guideline-based care for ACS, diabetes, and coronary heart disease risk factors (baseline characteristics of the treatment is described in JAMA 2014 cited above). Concomitant use of systemic corticosteroids for longer than 2 weeks, thiazolidinediones or fibrates was not permitted. Patients returned for outpatient visits at 1, 3, 6, 9 and 12 months following randomization, followed by alternating visits and phone contact every third month thereafter. The study drug was not interrupted until the repeated test of serum creatinine value was superior to 50% increase over the baseline visit.

In order to evaluate the renal function decline, the patients who had a linear GFR decline (defined as estimated GFR<80 ml/min/1.73 m² and UACR>300 mg/g) were selected for the analysis of the renal effect (UACR is Urine Albumin to Creatinine Ratio). The baseline mean blood pressure (BP) was 141/79 mmHg and 140/79 mmHg respectively for aleglitazar and placebo group and the mean BP level was stable during the treatment phase until the end of follow up. Surprisingly, after initial drop of eGFR (estimated glomular filtration rate), the mean eGFR slope (the rate of yearly GFR loss or the rate of yearly kidney function decline) in the chronic phase was stabilized over the next 18 months. The rate of kidney function loss was significantly slower in the aleglitazar group than that in the placebo group. The kidney function was then stabilized in the aleglitazar group whereas it continued to decrease in the placebo group.

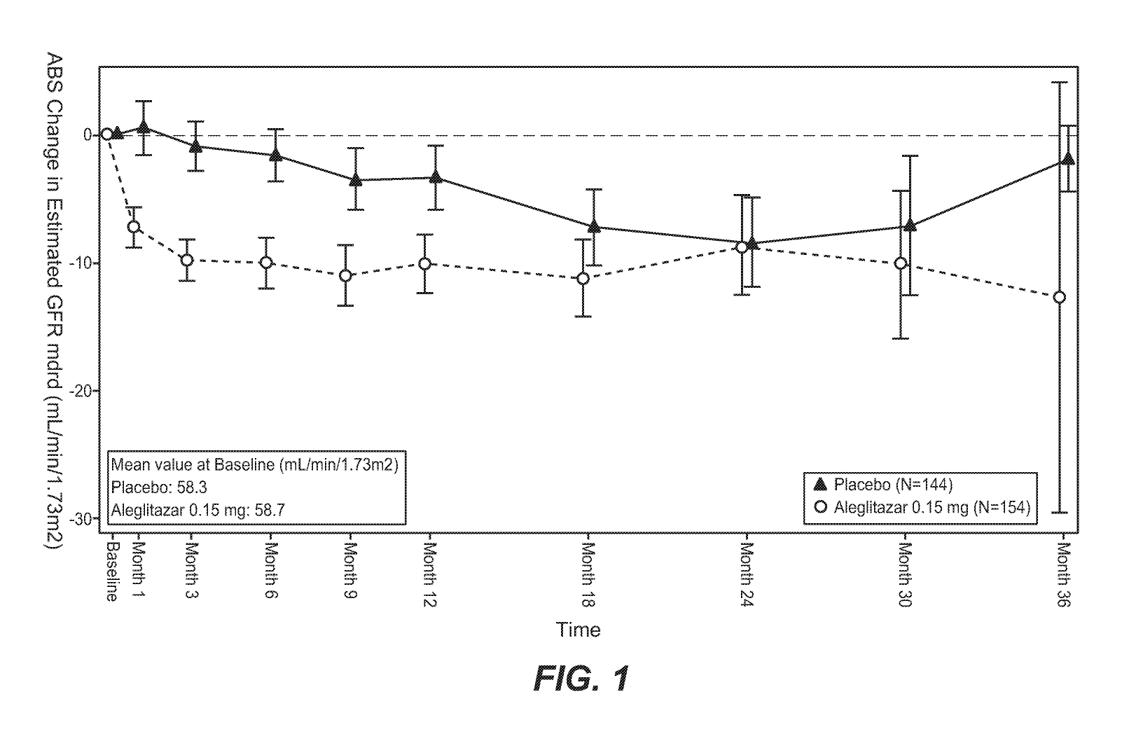

This is represented in FIG. 1.

FIG. 1 represents the eGFR slope analysis for patients with baseline eGFR inferior to 80 mL/min/1.73 m² and UACR superior to 300 mg/mg.

The patient number under each data point of the GFR slope of FIG. 1 is given in Table 1.

TABLE 1

| | | Month | | | | | | | | |
|---|---|---|---|---|---|---|---|---|---|---|
| Treatment group | Baseline | 1 | 3 | 6 | 9 | 12 | 18 | 24 | 30 | 36 |
| Placebo (N = 144) | 143 | 135 | 130 | 122 | 118 | 115 | 88 | 58 | 30 | 5 |
| Aleglitazar (N = 154) | 153 | 139 | 131 | 118 | 102 | 90 | 74 | 47 | 19 | 4 |

The patient number decreased significantly after month 24 due to the early termination of the AleCardio study, therefore only the results until month 24 can be considered.

In the subgroup of patients who have linear GFR decline (eGFR<80 mL/min/1.73 m² and UACR>300 mg/g), after initial drop of the eGFR, the rate of GFR loss in the aleglitazar treated group (79% on angiotensin-converting enzyme inhibitor (ACEi) or angiotensin receptor blocker (ARB)) was halted to a stable level; but the placebo group (79% on ACEi or ARB) continued to have progressive GFR drop. This is the first GFR slope analysis that shows the stabilization of renal function from a randomized placebo controlled trial in diabetic nephropathy patients.

The rate of kidney function decline (GFR loss) was over 4 mL/min/year in the placebo group (receiving standard of care).

The rate of kidney function decline became flat in the chronic phase in the aleglitazar group (receiving aleglitazar on top of standard of care).

The initial drop of GFR in the aleglitazar group was due to hemodynamic response, which is reversible after stopping the drug.

The invention thus relates to aleglitazar for use in the treatment or prevention of diabetic kidney disease in a patient having a linear decline in GFR.

Patients having a linear decline in GFR are patients having a progressive decline in renal function.

A linear decline in GFR is characterized by a negative slope of the curve plotting the absolute change in estimated GFR compared to baseline (Y axis) against time (X axis).

The invention further relates to alelgitazar for use as defined above wherein the patient has a GFR inferior to 80 mL/min/1.73 m² and macroalbumineria.

The invention further relates to aleglitazar for use as defined above wherein macroalbumineria is characterized by a UACR superior to 300 mg/g.

The invention thus relates to aleglitazar for use as defined above wherein the patient has a GFR inferior to 80 mL/min/1.73 m² and a UACR superior to 300 mg/g.

The invention also relates to aleglitazar for use as defined above wherein the treatment or prevention of diabetic kidney disease comprises the stabilization of kidney function and the prevention of end stage kidney disease.

End stage kidney disease occurs when the kidneys are no longer able to support the body's needs and work at a level needed for a day-to-day life.

The invention further relates to:

Aleglitazar for use as defined above, wherein the stabilization of kidney function is characterized by the halt of progressive kidney function loss;

Aleglitazar for use as defined above wherein the stabilization of kidney function is characterized by a null or positive slope of the curve plotting the absolute change in estimated GFR compared to baseline (Y axis) against time (X axis);

Aleglitazar for use as defined above wherein the stabilization of kidney function is characterized by a null or positive slope of the curve plotting the absolute change in estimated GFR compared to baseline (Y axis) against time (X axis) after 3 months of treatment with aleglitazar;

Aleglitazar for use as defined above wherein the stabilization of kidney function is characterized by a null or positive slope of the curve plotting the absolute change in estimated GFR compared to baseline (Y axis) against time (X axis) after 6 months of treatment with aleglitazar;

Aleglitazar for use as defined above wherein the stabilization of kidney function is characterized by a positive slope of the curve plotting the absolute change in estimated GFR compared to baseline (Y axis) against time (X axis) after 18 months of treatment with aleglitazar;

Aleglitazar for use as defined above wherein aleglitazar is for oral use;

Aleglitazar for use as defined above wherein aleglitazar is for use once a day;

Aleglitazar for use as defined above wherein aleglitazar is for use at a dose of 150 ug.

Aleglitazar for use as defined above wherein aleglitazar is for oral use at a dose of 150 ug once a day.

The invention further relates to:

The use of aleglitazar in the manufacture of a medicament for treating or preventing diabetic kidney disease in a patient having a linear decline in GFR;

The use of aleglitazar in the manufacture of a medicament for treating or preventing diabetic kidney disease in a patient, wherein the diabetic kidney disease is characterized by a linear decline in GFR;

The use of aleglitazar for the manufacture of a medicament for treating or preventing diabetic kidney disease, wherein the diabetic kidney disease is caused by a linear decline in GFR;

The use as defined above wherein the patient has a GFR inferior to 80 mL/min/1.73 m² and macroalbumineria;

The use as defined above wherein macroalbumineria is characterized by a UACR superior to 300 mg/g;

The use as defined above wherein the patient has a GFR inferior to 80 mL/min/1.73 m² and a UACR superior to 300 mg/g;

The use as defined above wherein the treatment or prevention of diabetic kidney disease comprises the stabilization of kidney function and the prevention of end stage kidney disease;

The use as defined above wherein the stabilization of kidney function is characterized by the halt of progressive kidney function loss;

The use as defined above wherein the stabilization of kidney function is characterized by a null or positive slope of the curve plotting the absolute change in estimated GFR compared to baseline (Y axis) against time (X axis);

The use as defined above wherein the stabilization of kidney function is characterized by a null or positive slope of the curve plotting the absolute change in estimated GFR compared to baseline (Y axis) against time (X axis) after 3 months of treatment with aleglitazar;

The use as defined above wherein the stabilization of kidney function is characterized by a null or positive slope of the curve plotting the absolute change in estimated GFR compared to baseline (Y axis) against time (X axis) after 6 months of treatment with aleglitazar;

The use as defined above wherein the stabilization of kidney function is characterized by a null or positive slope of the curve plotting the absolute change in estimated GFR compared to baseline (Y axis) against time (X axis) after 18 months of treatment with aleglitazar;

The use as defined above wherein the medicament comprises a dose of aleglitazar of 150 ug;

The use as defined above wherein the medicament is for oral administration;

The use as defined above wherein the medicament is for administration once a day;

The use as defined above wherein the medicament is for oral administration at a dose of 150 ug once a day;

A method for treating or preventing diabetic kidney disease in a patient in need thereof and having a linear decline in GFR comprising the administration of aleglitazar to the patient;

The method as defined above wherein the patient has a GFR inferior to 80 mL/min/1.73 m² and macroalbumineria; The method as defined above wherein macroalbumineria is characterized by a UACR superior to 300 mg/g;

The method as defined above wherein the patient has a GFR inferior to 80 mL/min/1.73 m² and a UACR superior to 300 mg/g;

The method as defined above wherein the treatment or prevention of diabetic kidney disease comprises the stabilization of kidney function and the prevention of end stage kidney disease;

The method as defined above wherein the stabilization of kidney function is characterized by the halt of progressive kidney function loss;

The method as defined above wherein the stabilization of kidney function is characterized by a null or positive slope of the curve plotting the absolute change in estimated GFR compared to baseline (Y axis) against time (X axis);

The method as defined above wherein the stabilization of kidney function is characterized by a null or positive slope of the curve plotting the absolute change in estimated GFR compared to baseline (Y axis) against time (X axis) after 3 months of treatment with aleglitazar;

The method as defined above wherein the stabilization of kidney function is characterized by a null or positive slope of the curve plotting the absolute change in estimated GFR compared to baseline (Y axis) against time (X axis) after 6 months of treatment with aleglitazar;

The method as defined above wherein the stabilization of kidney function is characterized by a null or positive slope of the curve plotting the absolute change in estimated GFR compared to baseline (Y axis) against time (X axis) after 18 months of treatment with aleglitazar;

The method as defined above wherein aleglitazar is administered at a dose of 150 ug;

The method as defined above wherein aleglitazar is orally administered;

The method as defined above wherein aleglitazar is administered once a day; and

The method as defined above wherein aleglitazar is orally administered once a day at a dose of 150 ug.

1. A method for the treatment or prevention of diabetic kidney disease in a patient having a linear decline in GFR, the method comprising administering to said patient an effective amount of aleglitazar.

2. The method of claim 1, wherein the patient has a GFR inferior to 80 mL/min/1.73 m² and macroalbumineria.

3. The method of claim 2, wherein macroalbumineria is characterized by a UACR superior to 300 mg/g.

4. The method of claim 3, further comprising stabilizing kidney function and preventing end stage kidney disease.

5. The method of claim 4, wherein the stabilizing of kidney function is characterized by the halt of progressive kidney function loss.

6. The method of claim 5, wherein the stabilizing of kidney function is characterized by a positive slope of the curve plotting the absolute change in estimated GFR compared to baseline (Y axis) against time (X axis).

7. The method of claim 6, wherein the stabilizing of kidney function is characterized by a positive slope of the curve plotting the absolute change in estimated GFR compared to baseline (Y axis) against time (X axis) after 3 months of treatment with aleglitazar.

8. The method of claim 7, wherein the aleglitazar is orally administered at a dose of 150 ug once a day.
